(19) United States
(12) Patent Application Publication
(10) Pub. No.: US 2011/0034711 A1
(43) Pub. Date: Feb. 10, 2011

(54) TITANOSILICATE

(75) Inventors: Tomonori Kawabata, Osaka (JP); Hiroaki Abekawa, Osaka (JP)

Correspondence Address: FOLEY AND LARDNER LLP;SUITE 500, 3000 K STREET NW, WASHINGTON, DC, 20007, US

(73) Assignee: Sumitomo Chemical Company, Limited

(21) Appl. No.: 12/937,994

(22) PCT Filed: Mar. 30, 2009
(86) PCT No.: PCT/JP2009/057011

§ 371 Date: Oct. 14, 2010

(30) Foreign Application Priority Data
Apr. 17, 2008 (JP) 2008-107630

(51) Int. Cl. B01J 29/89; B01J 21/06; C01B 39/02; C01B 33/20; C07D 301/03

(52) U.S. Cl. 549/523; 502/242; 502/60; 423/326; 423/700

(57) ABSTRACT

A layered titanosilicate obtained by contacting a layered borosilicate with a titanium source and an inorganic acid, and a method for producing an epoxy compound wherein the layered titanosilicate is used as a catalyst.

### TECHNICAL FIELD

The present invention relates to a titanosilicate, improvement thereof and a method for producing the same.

### BACKGROUND ART

The titanosilicates are known as effective catalysts for the production of epoxy compounds by epoxidation of olefins, the production of phenol compounds or polyhydroxyphenyl compounds by hydroxylation reactions of benzene or phenol compounds. It is known that a 4-coordinated titanium species present in the silicate framework is the active site.

As a general method for producing such titanosilicates, there is known a method where, using a surfactant as a templating agent or structure-directing agent, a titanium compound and a silicon compound are hydrolyzed and, if necessary, after crystallization or improvement of pore regularity by hydrotheimal synthesis or the like, the surfactant is removed by calcination or extraction (for example, Non-Patent Document 1); or a method where a crystalline silicate subjected to deboronization treatment, a titanium compound, water and a structure-directing agent are blended and, thereafter, heat-treated (Non-Patent Document 2).

However, according to the method of Non-Patent Document 1, there occurs a phenomenon that the titanium species precipitates outside the silicate framework, in addition to the 4-coordinated titanium species, and becomes a cause for deterioration of activity. Therefore, it is required to wash and remove the excessive titanium species in the presence of a strong acid. However, in the presence of a strong acid, there has been a problem that generation of titanium species outside the silicate framework also occurs. The method of Non-Patent Document 2 is a method to solve the problem of generation of the titanium species outside the silicate framework, but operations involved are very cumbersome and a large number of preparation steps are needed, making the method unfavorable in terms of cost.

Further, as a method to obtain a titanosilicate where titanium is incorporated completely in the lattice, there is reported a method where a β-zeolite synthesized with or without use of titanium is treated with a solution containing an inorganic acid in the presence of a titanium source (Patent Document 1). However, this method is a method where titanium is introduced into the lattice by dealumination of the zeolite containing aluminum in its structure and had a problem that, even when the aluminum content in the structure of titanium zeolite synthesized is decreased, selectivity for epoxide formation was not necessarily sufficient when used in the epoxidation reaction (see Patent Document 1).
[Patent Document 1] JP No. H11-510136 T
[Non-Patent Document 1] Journal of Physical Chemistry B, 105, 2897-2905 (2001)
[Non-Patent Document 2] Chemical Communications, 1026-1027 (2002)

### DISCLOSURE OF THE INVENTION

The present invention provides a titanosilicate which can be obtained by a convenient method and shows an excellent catalytic activity, a method for producing the same, and a method for producing an epoxy compound using the titanosilicate as a catalyst.

That is, the present invention relates to a layered titanosilicate obtained by contacting a layered borosilicate with a solution containing a titanium source and an inorganic acid; a titanosilicate having a zeolite structure (hereinafter, also referred to as the titanosilicates of the present invention), obtained by heat treatment of the layered titanosilicate; a method for producing the titanosilicates of the present invention; and, further, a method for producing an epoxy compound, characterized in that an olefin, oxygen and hydrogen are reacted in the presence of the titanosilicates of the present invention and a noble metal catalyst.

The titanosilicates of the present invention not only show excellent catalytic activity but also have superior selectivity in the epoxidation reaction of olefins by use of hydrogen peroxide, or oxygen and hydrogen. Further, in contrast to the β-zeolite, a titanosilicate wherein titanium is incorporated completely into the lattice cannot be obtained in case of a zeolite prepared via a layered silicate, even when the same, after being transformed into a zeolite, is treated with a solution containing an inorganic acid in the presence of a titanium source. However, according to the method of the present invention, the desired titanosilicate can be obtained. Moreover, the layered titanosilicate obtained can be transformed into a zeolite easily by heat treatment and the resultant titanosilicate having a zeolite structure also shows excellent catalytic activity and selectivity in the epoxidation reaction of olefins.

### BRIEF DESCRIPTION OF THE DRAWINGS

FIG. 1 is a graph showing the results of measurement of the UV-visible absorption spectra of titanosilicates obtained in Example 1 and Comparative Examples 1 and 2.

### BEST MODES FOR CARRYING OUT THE INVENTION

The layered borosilicate used in the present invention refers to one of layered silicates having two-dimensional layered framework structures comprising covalent bonds between Si (silicon) and O (oxygen), wherein a part of Si in the SiO₂ framework is replaced with boron. The layered borosilicate, usually, has a composition represented by the general formula: xB₂O₃.(1-x)SiO₂ (in the formula, x represents a number from 0.0001 to 0.5) and, especially, a layered borosilicate wherein x is from 0.01 to 0.2 is suitable. As such a layered borosilicate, for example, there may be cited a precursor of B-MWW (for example, one described in J. Phys. Chem. B, 105, 2897-2905 (2001)), which is a crystalline borosilicate having a MWW structure according to the IZA (International Zeolite Association) structural code and contains B in the SiO₂ framework.

As the inorganic acid in the present invention, an inorganic acid having higher oxidation-reduction potential than the tetra-valent titanium is preferable and is exemplified by nitric acid, perchloric acid, fluorosulfonic acid, and a mixture of these. The concentration of the inorganic acid used is not particularly limited and can be in a range of 0.01 M to 20 M (M: mol/l). The preferable concentration of the inorganic acid is 1 M to 5 M.

The titanium source in the present invention includes titanium compounds. The titanium compounds are exemplified by titanium alkoxides, titanium acetate, titanium nitrate, titanium sulfate, titanium phosphate, titanium perchlorate, titanium halides such as titanium tetrachloride, and titanium dioxide, with titanium alkoxides being especially preferable. Regarding the amount of titanium source to be used, an effect can be expected if its weight, as a titanium compound, is 0.001 time to 10 times the weight of the layered borosilicate, the preferable range being 0.01 time to 2 times.

Contact of the layered borosilicate with the titanium source and inorganic acid is usually carried out by making the layered borosilicate contact with a mixture of the titanium source and inorganic acid. The temperature for this is preferably 20° C. to 150° C., and, further, an especially preferable temperature range is 50° C. to 104° C. The pressure at the time of contact is not particularly limited but is usually about 0 to 10 MPa in gauge pressure. By treating the layered borosilicate under these conditions, a layered titanosilicate can be obtained. The obtained layered titanosilicate can be further transformed into a (crystalline)titanosilicate having a zeolite structure (for example, a titanosilicate having an MWW structure) by subjecting the same to interlayer dehydration condensation to form a zeolite structure (Chemistry Letters, 774-775 (2000)). The dehydration condensation is carried out by heat treatment (hereinafter, also referred to as the heat treatment), for example, by heating to about 800° C.

The obtained layered titanosilicate and titanosilicate having a zeolite structure belong to a group of compounds, collectively termed titanosilicates. A term “titanosilicates” is a collective term for porous silicates (SiO₂), wherein a part of Si is replaced with Ti. Ti in titanosilicate is incorporated into the SiO₂ framework and it can be confirmed easily that Ti is incorporated into the SiO₂ framework by the presence of a peak at 210 nm to 230 nm in the UV-visible spectra. Also, in contrast to Ti in TiO₂, which is usually 6-coordinated, Ti in the titanosilicate is 4-coordinated and, thus, can be confirmed easily by measuring the coordination number by a titanium K-edge XAFS analysis and the like. One of the characteristics of the titanosilicates of the present invention, as compared to the heretofore known titanosilicates, is that they have more absorption at around 210 nm to 230 nm, which represents the 4-coordinated Ti species, and less absorption at 320 nm to 330 nm, which represents the Ti species outside the framework.

Among the titanosilicates synthesized according to the method of the present invention, a crystalline titanosilicate or layered titanosilicate having a composition represented by the general formula: xTiO₂.(1-x)SiO₂ (in the formula, x represents a number from 0.0001 to 0.1) and having pores not smaller than a 12-membered oxygen ring, is preferable from a viewpoint of catalytic performance in the epoxidation reaction of olefins. Examples of a crystalline titanosilicate having pores not smaller than a 12-membered oxygen ring include the layered titanosilicates obtained according to the aforementioned method of the present invention and Ti-MWW obtained by calcination of the same. These titanosilicates are the titanosilicates which possess the following X-ray diffraction pattern, just like the heretofore known Ti-MWW, a Ti-MWW precursor (for example, the Ti-MWW precursor described in Japanese Patent Laid-Open No. 2003-327425) and. Ti-YNU-1 (for example, Ti-YNU-1 described in Angewandte Chemic International Edition 43, 236-240, (2004)).

X-ray diffraction pattern:

(Lattice spacing d/Å)

12.3±0.3

11.0±0.3

9.0±0.3

6.1±0.3

3.9±0.2

3.4±0.1

The titanosilicates synthesized in the present invention may be silylated with a silylating agent such as, 1,1,1,3,3,3-hexamethyldisilazane and used as a catalyst in the epoxidation reaction of olefins. By silylation, the catalytic activity and reaction selectivity can be made even higher.

Olefins involved in the present invention include, for example, ethylene, propylene, butene, pentene, hexene, heptene, octene, nonene, decene, 2-butene, isobutene, 2-pentene, 3-pentne, 2-hexene, 3-hexycene, 4-methyl-1-pentene, 2-heptene, 3-heptene, 2-okutene, 3-octene, 2-nonene, 3-nonene, 2-decene and 3-decene.

The thus obtained layered titanosilicate or (crystalline)titanosilicate having a zeolite structure obtained by heat treatment of the same, or those obtained by silylating these, namely the silylated layered titanosilicate or silylated crystalline titanosilicate (hereinafter, the titanosilicates of the present invention and their silylated derivatives are referred to as the titanosilicates and the like of the present invention) can be used, together with a noble metal catalyst, as a catalyst for a reaction to produce an epoxy compound by reacting an olefin, oxygen and hydrogen.

When propylene is used as the olefin, the titanosilicate and the like of the present invention can be used as a catalyst for the reaction to produce propylene oxide by reacting oxygen and hydrogen together with propylene and a noble metal catalyst (hereinafter, referred to as the propylene oxide production reaction). Hereinafter, a method for producing propylene oxide according to the present invention will be described.

The titanosilicates and the like of the present invention can also be used after activation by treatment of contacting with a hydrogen peroxide solution of an adequate concentration. Usually, the concentration of hydrogen peroxide solution can be selected in a range of 0.0001% by weight to 50% by weight. The solvent of the hydrogen peroxide solution is not particularly limited but water or the solvent used in the synthetic reaction of propylene oxide is industrially convenient and preferable.

The temperature of the treatment by hydrogen peroxide is selected usually in a range of 0° C. to 100° C. Preferable temperature is 0° C. to 60° C. Further, the treatment time is, although it depends on the concentration of hydrogen peroxide, usually 10 minutes to 5 hours, preferably 1 hour to 3 hours.

The noble metals used in the propylene oxide production reaction include palladium, platinum, ruthenium, rhodium, iridium, osmium, gold, and an alloy or mixture of these. Preferable noble metals include palladium, platinum and gold. Further, more preferable noble metal is palladium. As the palladium, for example, a palladium colloid may be used (see, for example, JP No. 2002-294301 A, Example 1. and the like). The palladium can be used with metals such as platinum, gold, rhodium, iridium and osmium added and mixed. The preferable metal to be added is platinum. In addition, these noble metals may be in a state of compound such as oxide and hydroxide. These may be charged in the reactor in a state of noble metal compound and, under the reaction condition, they may be partially or wholly reduced by hydrogen included in the raw materials for the reaction.

The noble metals are usually used supported on carriers. The noble metals can be used supported on titanosilicates or they may be used supported on other carriers than titanosilicates, including oxides such as silica, alumina, titania, zirconia and niobia; hydrates such as niobic acid, zirconic acid, tungstic acid and titanic acid; or carbons and mixtures thereof When the noble metals are supported on carriers other than the titanosilicates, the carriers supporting the noble metals may be mixed with the titanosilicates and the mixtures may be used as catalysts. Among the carriers other than titanosilicates, carbons are mentioned as preferable carriers. As the carbonaceous carriers, there are known activated carbon, carbon black, graphite and carbon nanotubes.

As a preparative method for the noble metal-supported catalyst, for example, there is known a method where an ammine complex such as Pd tetraammine chloride is supported on a carrier by an impregnation method or the like, followed by reduction. The method for reduction may include reduction using a reducing agent such as hydrogen or reduction using an ammonia gas evolved at the time of thermal decomposition under inert gas. The temperature of reduction depends on the kind of the noble metal ammine complex, but when Pd tetraammine chloride is used, the temperature is usually 100° C. to 500° C., preferably 200° C. to 350° C.

The thus obtained noble metal-supporting material usually contains the noble metal in a range of 0.01 to 20% by weight, preferably 0.1 to 5% by weight. When used for the reaction, the weight ratio of the noble metal to the titanosilicate (weight of noble metal/weight of titanosilicate) is preferably 0.01 to 100% by weight, more preferably 0.1 to 20% by weight.

The propylene oxide producing reaction using the titanosilicates and the like of the present invention as a catalyst is usually carried out in a liquid phase comprising a mixed solvent of a nitrile compound and water. Suitable nitrile compounds include a linear- or branched-chain saturated aliphatic nitrile or aromatic nitrile. These nitrile compounds are exemplified by C₂ to C₄ alkylnitriles such as acetonitrile, propionitrile, isobutyronitrile and butyronitrile; and benzonitrile. Among these, acetonitrile is preferable.

Usually, the ratio of water and the nitrile compound is, in a weight ratio, 90:10 to 0.01:99.99, preferably 50:50 to 0.01:99.99. If the ratio of water becomes too high, there are cases where propylene oxide becomes liable to deteriorate by ring-opening due to a reaction with water, and there are also cases where selectivity for propylene oxide becomes lower. Conversely, if the ratio of the nitrile compound becomes too high, the cost for solvent recovery becomes high.

In the reaction to produce propylene oxide, a method in which a buffer salt is added to the reaction solvent is effective in preventing decrease in catalytic activity and in further increasing catalytic activity and, thereby, improving efficiency of hydrogen use. The buffer salt may be used together with the noble metal or may be used each independently. The amount of the buffer salt to be added is usually 0.001 mmol/kg to 100 mmol/kg based on unit weight of the solvent (total weight of water and the organic solvent).

The buffer salt is exemplified by a buffer salt comprising 1) an anion selected from a sulfate ion, hydrogensulfate ion, carbonate ion, hydrogencarbonate ion, phosphate ion, hydrogenphosphate ion, dihydrogenphosphate ion, hydrogenpyrophosphate ion, pyrophosphate ion, halide ion, nitrate ion, hydroxide ion or C₁ to C₁₀ carboxylate ions, and 2) a cation selected from an ammonium, alkylammonium, alkylarylammonium, alkali metal or alkaline earth metal salt. The C₁ to C₁₀ carboxylate ions include a formate ion, an acetate ion, a propionate ion, a butyrate ion, a valerate ion, a caproate ion, a caprylate ion, a caprate ion and a benzoate ion.

Examples of alkylammonium include tetramethylammonium, tetraethylammonium, tetra-n-propylammonium, tetra-n-butylammonium and cetyltrimethylammonium. Examples of alkali metal or alkaline earth metal cations include a lithium cation, a sodium cation, a potassium cation, a rubidium cation, a cesium cation, a magnesium cation, a calcium ion, a strontium cation and a barium cation.

Preferable buffer salts include ammonium salts of inorganic acid such as ammonium sulfate, ammonium hydrogensulfate, ammonium carbonate, ammonium hydrogencarbonate, diammonium hydrogenphosphate, ammonium dihydrogenphosphate, ammonium phosphate, ammonium hydrogenpyrophosphate, ammonium pyrophosphate, ammonium chloride and ammonium nitrate, or ammonium salts of C₁ to C₁₀ carboxylic acids such as ammonium acetate. As a preferable ammonium salt, there may be mentioned ammonium dihydrogenphosphate.

In the propylene oxide production reaction of the present invention, a method in which a quinoid compound is added to the reaction solvent, together with the titanosilicates and the like of the present invention and a noble metal catalyst-supporting material, is also effective because it can further increase selectivity for propylene oxide.

The quinoid compounds are exemplified by p-quinoid compounds represented by the following formula (1) and phenanthraquinone compounds:

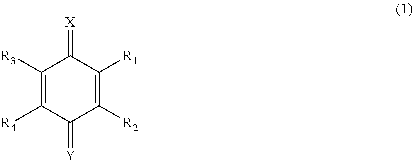

[In the formula (1), R₁, R₂, R₃, and R₄ represent a hydrogen atom; or each neighboring R₁ and R₂ or R₃ and R₄ each independently are linked together at both ends and, together with the carbon atoms of the quinone skeleton to which they are bonded, form a benzene ring or naphthalene ring, both of which may be substituted with an alkyl group or hydroxyl group; X and Y may be the same or different from each other, and represent an oxygen atom or NH group.]

The compounds represented by the formula (1) include:

1) a quinone compound (1A) represented by the formula (1), wherein R₁, R₂, R₃, and R₄ represent a hydrogen atom, and both X and Y represent an oxygen atom;

2) a quinone imine compound (1B) represented by the formula (1), wherein R₁, R₂, R₃, and R₄ represent a hydrogen atom, X represents an oxygen atom, and Y represents an NH group;

3) a quinone diimine compound (1C) represented by the formula (1), wherein R₁, R₂, R₃, and R₄ represent a hydrogen atom, and X and Y represent an NH group.

The quinoid compounds represented by the formula (1) include anthraquinone compounds represented by the following formula (2):

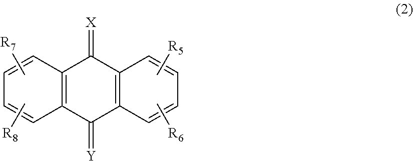

[In the formula (2), X and Y are as defined in the formula (1); R₅, R₆, R₇, and R₈ may be the same or different from each other and represent a hydrogen atom, hydroxyl group, or alkyl group (for example, a C₁ to C₅ alkyl group such as methyl, ethyl, propyl, butyl and pentyl).]

In the formulae (1) and (2), X and Y preferably represent an oxygen atom. The quinoid compounds represented by the formula (1), wherein X and Y are oxygen atoms, are especially referred to as quinone compounds or p-quinone compounds. Also, the quinoid compounds represented by the formula (2), wherein X and Y are oxygen atoms, are further especially referred to as anthraquinone compounds.

Dihydro derivatives of the quinoid compounds include the compounds represented by the formulae (3) and (4), which are the dihydro derivatives of the compounds represented by the formulae (1) and (2):

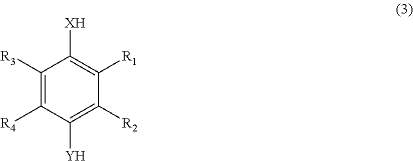

[In the formula (3), R₁, R₂, R₃, and R₄, X, and Y are as defined in relation to the formula (1)];

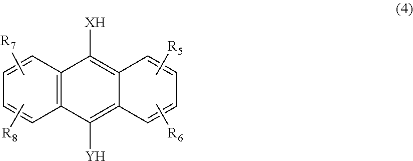

[In the formula (4), X, Y, R₅, R₆, R₇, and R₈ are as defined in relation to the formula (2).]

In the formulae (3) and (4), X and Y preferably represent an oxygen atom. The dihydro derivatives of the quinoid compounds represented by the formula (3), wherein X and Y are oxygen atoms, are especially referred to as dihydroquinone compounds or p-dihydroquinone compounds. Also, the dihydro derivatives of the quinoid compounds represented by the formula (4), wherein X and Y are oxygen atoms, are further especially referred to as dihydroanthraquinone compounds.

Examples of the phenanthraquinone compounds include 1,4-phenanthraquinone which is a p-quinoid compound, and 1,2-, 3,4-, and 9,10-phenathraquinones which are o-quinoid compounds.

Specific quinone compounds include benzoquinones, naphthoquinones, and anthraquinones; 2-alkylanthraquinone compounds such as 2-ethylanthraquinone, 2-t-butylanthraquinone, 2-amylanthraquinone, 2-methylanthraquinone, 2-butylanthraquinone, 2-t-amylanthraquinone, 2-isopropylanthraquinone, 2-s-butylanthraquinone or 2-s-amylanthraquinone; 2-hydroxyanthraquinone; polyalkylanthraquinone compounds such as 1,3-diethylanthraquinone, 2,3-dimethylanthraquinone, 1,4-dimethylanthraquinone or 2,7-dimethylanthraquinone; polyhydroxyanthraquinones such as 2,6-dihydroxyanthraquinone; and naphthoquinone and its mixtures. Preferable quinoid compounds include anthraquinone and 2-alkylanthraquinone compounds (in the formula (2), X and Y are oxygen atoms; R₅ is an alkyl group substituted at the 2-position; R₆, R₇, and R₈ represent a hydrogen atom). Preferable dihydro derivatives of quinoid compounds include the dihydro derivatives corresponding to these preferable quinoid compounds.

The method for adding the quinoid compound or dihydro derivative of the quinoid compound (hereinafter, abbreviated as the quinoid compound derivative) to the reaction solvent includes a method whereby the quinoid compound derivative is dissolved in the liquid phase and, thereafter, used for the reaction. For example, the hydrogenated compound of the quinoid compound such as hydroquinone or 9,10-anthracenediol may be added to the liquid phase and used by generating a quinoid compound in the reactor by oxidation with oxygen.

Further, the quinoid compounds used in the present invention, including the quinoid compounds exemplified above, may be partially transformed into dihydro derivatives, which are hydrogenated quinoid compounds, depending on the reaction conditions. These compounds may also be used.

The amount of the quinoid compound derivative used is selected usually in a range of 0.001 mmol/kg to 500 mmol/kg based on unit weight of the solvent (unit weight of water, organic solvent, or a mixture of both). The preferable amount of the quinoid compound is 0.01 mmol/kg to 50 mmol/kg.

Further, in the method of the present invention, the salts comprising ammonium, alkylammonium or alkylarylammonium and the quinoid compound can be added to the reaction system concurrently.

The methods for reaction in the propylene oxide production include a flowing fixed-bed reaction, flowing slurry complete mixing reaction, and the like.

The ratio of partial pressures of oxygen and hydrogen fed to the reactor is selected usually in a range of 1:50 to 50:1. A preferable ratio of the partial pressures of oxygen and hydrogen is 1:2 to 10:1. When the ratio of the partial pressures of oxygen and hydrogen (oxygen/hydrogen) is too high, there are cases where the production rate of propylene oxide is lowered. Also, when the ratio of the partial pressures of oxygen and hydrogen (oxygen/hydrogen) is too low, there are cases where selectivity for propylene oxide is lowered because of increase in propane generation as a byproduct. The reaction can be carried out with the oxygen and hydrogen gas used in the present reaction diluted with diluent gas. The diluent gas includes nitrogen, argon, carbon dioxide, methane, ethane and propane. There is no particular restriction on the concentration of the diluent gas, but the reaction is carried out with oxygen and hydrogen diluted according to necessity.

Oxygen sources include oxygen gas, air, and the like. As the oxygen gas, oxygen gas produced by an economical pressure swing method can be used and, if needed, high-purity oxygen gas produced by cryogenic separation can also be used.

The reaction temperature in the propylene oxide production reaction is usually 0° C. to 150° C., preferably 40° C. to 90° C. If the reaction temperature is too low, the rate of reaction becomes slow and, if the reaction temperature becomes too high, byproducts due to side reactions increase.

The reaction pressure is not particularly limited but is usually 0.1 MPa to 20 MPa, preferably 1 MPa to 10 MPa, in gauge pressure. If the pressure is too low, dissolution of the raw material gas becomes insufficient and the rate of reaction becomes slow. If the reaction pressure is too high, the cost of the equipments involved in the reaction increases.

Recovery of propylene oxide, the product of the reaction, can he carried out by the usual distillative separation. Heretofore, production of propylene oxide has been described as an example, but the above-described production method is applicable as the production method for producing epoxy compounds using olefins other than propylene. The epoxy compounds other than propylene oxide include, for example, ethylene oxide, butene oxide and pentene oxide.

### Examples

Hereinafter, the present invention will be described in terms of Examples, but the present invention is not limited to these Examples.

### Example 1

### [Preparation of Layered Borosilicate]

In an autoclave, a gel was prepared by dissolving under stirring 162 g of boric acid and 117 g of fumed silica (cab-o-sil M7D) in 257 g of piperidine and 686 g of purified water at room temperature under an air atmosphere and, after aging for 1.5 hours, the autoclave was closed tightly. After raising the temperature over 8 hours under further stirring, a hydrothermal synthesis was carried out by maintaining at 165° C. for 120 hours to obtain a suspended solution. The suspended solution was filtered and washed until the pH of the filtrate became about 10. Then, the filter cake was dried at 50° C. to obtain 120 g of white powder. The X-ray diffraction pattern of this white power was measured by the use of an X-ray diffraction apparatus using copper K-α radiation. As a result, the white powder was confirmed to be a layered borosilicate. Also, according to an ICP emission analysis, the boron content was 1.1% by weight and silicon content was 32.7% by weight.

[Ti-MWW Precursor Catalyst (Titanosilicate Catalyst A)]

To 15 g of the layered borosilicate obtained as described above was added 777 g of 2 N nitric acid and 1.9 g of TBOT (tetra-n-butylorthotitanate), followed by reflux for 20 hours. Thereafter, by filtration, washing until neutral, and vacuum drying at 150° C., 12 g of white powder was obtained. The X-ray diffraction pattern of this white power was measured by the use of an X-ray diffraction apparatus using copper K-α radiation. As a result, the white powder was confirmed to be a Ti-MWW precursor. Also, the titanium content according to an ICP emission analysis was 0.94% by weight.

[Ti-MWW Catalyst (Titanosilicate Catalyst B)]

The Ti-MWW precursor obtained (Titanosilicate Catalyst A, 10 g) was calcinated at 530° C. for 6 hours to obtain 9 g of Ti-MWW catalyst powder. It was confirmed that the powder obtained had an MWW structure by measuring an X-ray diffraction pattern. In addition, the titanium content according to an ICP emission analysis was 1.01% by weight.

[Pd/Activated Carbon (AC) Catalyst 1]

A Pd/activated carbon (AC) catalyst was prepared by the following method. In a 500 mL recovery flask was prepared a 300 mL aqueous solution containing 0.30 mmol of a palladium colloid (produced by JGC Catalysts and Chemicals Ltd.). To this aqueous solution was added 3 g of activated carbon (produced by Wako Pure Chemical Ind., Ltd.) and the mixture was stirred for 8 hours at room temperature. After completion of stirring, water was removed by use of a rotary evaporator, vacuum dried at 80° C. for 6 hours and, further, calcinated at 300° C. for 6 hours under a nitrogen atmosphere to obtain a Pd/AC catalyst (Pd/AC Catalyst 1).

[Pd/Activated Carbon (AC) Catalyst 2]

In a 500 mL recovery flask was prepared a 300 mL aqueous solution containing 0.30 mmol of Pd tetraammine chloride. To this aqueous solution was added 3 g of activated carbon (produced by Wako Pure Chemical Ind., Ltd.) and the mixture was stirred for 8 hours. After completion of stirring, water was removed by use of a rotary evaporator and, further, vacuum dried at 80° C. for 6 hours. The catalyst precursor powder obtained was calcinated at 300° C. for 6 hours under a nitrogen atmosphere to obtain a Pd/AC catalyst (Pd/AC Catalyst 2).

### Example 1-1

### Production of Propylene Oxide (Condition 1)

In an autoclave with a volume of 0.3 L were charged 131 g of acetonitrile-water having a weight ratio of water/acetonitrile=30/70, 2.28 g of Titanosilicate Catalyst A treated beforehand with hydrogen peroxide, and 0.198 g of Pd/AC Catalyst 1. Thereafter, the pressure was adjusted to 4 MPa in absolute pressure with nitrogen and the temperature inside the autoclave was adjusted to 50° C. by circulating warm water through the jacket. To the autoclave were continuously fed mixed gas comprising 3.6% by volume of hydrogen, 2.1% by volume of oxygen, and 94.3% by volume of nitrogen at a rate of 146 NL/hr; acetonitrile-water (with the weight ratio of water/acetonitrile being 30/70) comprising 0.7 mmol/kg of anthraquinone and 0.7 mmol/kg of ammonium dihydrogenphosphate at a rate of 90 g/Hr; and liquid propylene containing 0.4% by volume of propane at a rate of 36 g/Hr. During the reaction, the reaction temperature was controlled at 50° C. and the reaction pressure at 4 MPa. The liquid component and gas component were continuously withdrawn, with the solid components, the Titanosilicate Catalyst A and Pd/AC Catalyst 1, filtered off by a sintered filter, gas and liquid separated, and, thereafter, the pressure returned to normal. After 6 hours, samples of the reaction liquid and gas were taken simultaneously, and the liquid and gas were each analyzed by gas chromatography. The amount of propylene oxide produced was 61.1 mmol/Hr and selectivity for propylene glycol {(amount of propylene glycol produced)/(amount of propylene oxide produced+amount of propylene glycol produced)×100} was 5.6% (Table 1).

### Example 1-2

### Production of Propylene Oxide (Condition 2)

Using an autoclave with a volume of 0.5 L as a reactor for the reaction, therein were fed raw material gas comprising propylene/oxygen/hydrogen/nitrogen in a volume ratio of 4/4/10/82 at a rate of 16 L/Hr and a solution of water/acetonitrile=20/80 (weight ratio) comprising 0.7 mmol/kg of anthraquinone at a rate of 108 mL/Hr. Then, by withdrawing the reaction mixture from the reactor through a filter, a continuous reaction was carried out under conditions of temperature, 60° C.; pressure, 0.8 MPa (gauge pressure); and residence time, 90 minutes. During this period, in the reaction mixture inside the reactor, 131 g of the reaction solvent, 0.266 g of Titanosilicate Catalyst A treated beforehand with hydrogen peroxide, and 0.03 g of Pd/AC Catalyst 2 were made to be present,. The liquid phase and gas phase withdrawn after 5 hours from the start of the reaction were analyzed by gas chromatography. As a result, the amount of propylene oxide produced was 5.48 mmol/Hr and selectivity for propylene glycol was 2.5% (Table 2).

### Example 1-3

### Production of Propylene Oxide (Condition 3)

Production of propylene oxide was conducted by carrying out the same operation as in Condition 2, except that Titanosilicate Catalyst B was used instead of Titanosilicate Catalyst A. After 6 hours, the liquid phase and gas phase were each analyzed by gas chromatography. As a result, the amount of propylene oxide produced was 5.58 mmol/Hr and selectivity for propylene glycol was 3.0% (Table 2).

### Comparative Example 1

### Ti-MWW Precursor Catalyst (Titanosilicate Catalyst C)

In an autoclave, a gel was prepared by dissolving under stirring 112 g of TBOT (tetra-n-butylorthotitanate), 565 g of boric acid, and 410 g of fumed silica (cab-o-sil M7D) in 899 g of piperidine and 2402 g of purified water at room temperature under an air atmosphere and, after aging for 1.5 hours, the autoclave was closed tightly. After raising the temperature over 8 hours under further stirring, a hydrothermal synthesis was carried out by maintaining the reaction mixture at 160° C. for 120 hours to obtain a suspended solution. The suspended solution was filtered and, thereafter, washed until the pH of the filtrate became about 10. Then, the filter cake was dried at 50° C. to obtain 540 g of white powder. To 15 g of the powder obtained was added 750 ml of 2 N nitric acid, followed by reflux for 20 hours. By subsequent filtration, washing until nearly neutral, and drying sufficiently at 50° C., 11 g of white powder was obtained. The X-ray diffraction pattern of this white power was measured by the use of an X-ray diffraction apparatus using copper K-α radiation. As a result, the white powder was confirmed to be a Ti-MWW precursor and the titanium content according to an ICP emission analysis was 1.65% by weight.

Production of propylene oxide was conducted by carrying out the same operation as in Example 1 according to Condition 1, except that Titanosilicate Catalyst C was used instead of Titanosilicate Catalyst A. After 6 hours, the liquid phase and gas phase were each analyzed by gas chromatography. As a result, the amount of propylene oxide produced was 60.5 mmol/Hr and selectivity for propylene glycol was 15.7% (Table 1).

TABLE 1

| Example/ | | PO | PG production | PG selectivity/ |
|---|---|---|---|---|
| Comparative | | production | rate | % PG/ |
| Example | Catalyst | rate mmol/h | mmol/h | (PO + PG) |
| Example 1-1 | Catalyst A | 61.1 | 3.60 | 5.6 |
| Comparative | Catalyst C | 60.5 | 11.3 | 15.7 |
| Example 1 | | | | |

### Condition 1

### Comparative Example 2

### Ti-MWW Catalyst (Titanosilicate Catalyst D)

To 45 g of the layered borosilicate prepared in Example 1 was added 2250 mL of 2 N nitric acid and was heated under reflux for 20 hours. By subsequent filtration, washing until nearly neutral, and drying sufficiently at 50° C., 33 g of white powder was obtained. The powder obtained was calcinated at 530° C. for 6 hours to obtain powder having an MWW structure. It was confirmed that the obtained powder had an MWW structure by measuring an X-ray diffraction pattern. Further, to 15 g of the powder having an MWW structure was added 777 g of 2 N nitric acid and 1.9 g of TBOT (tetra-n-butylorthotitanate), followed by heating under reflux for 20 hours. By subsequent filtration, washing until nearly neutral, and vacuum drying at 150° C., 11 g of white powder was obtained. The X-ray diffraction pattern of this white power was measured by the use of an X-ray diffraction apparatus using copper K-α radiation. As a result, the white powder was confirmed to be Ti-MWW and the titanium content according to an ICP emission analysis was 0.36% by weight.

Production of propylene oxide was conducted by carrying out the same operation as in Example 1 according to Condition 2, except that Titanosilicate Catalyst D was used instead of Titanosilicate Catalyst A. After 6 hours, the liquid phase and gas phase were each analyzed by gas chromatography. As a result, the amount of propylene oxide produced was 0.08 mmol/Hr and selectivity for propylene glycol was 20.0% (Table 2).

TABLE 2

| Example/ | | PO | PG production | PG selectivity/ |
|---|---|---|---|---|
| Comparative | | production | rate | % PG/ |
| Example | Catalyst | rate mmol/h | mmol/h | (PO + PG) |
| Example 1-2 | Catalyst A | 5.48 | 0.14 | 2.5 |
| Example 1-3 | Catalyst B | 5.58 | 0.17 | 3.0 |
| Comparative | Catalyst D | 0.08 | 0.02 | 20.0 |
| Example 2 | | | | |

### Condition 2

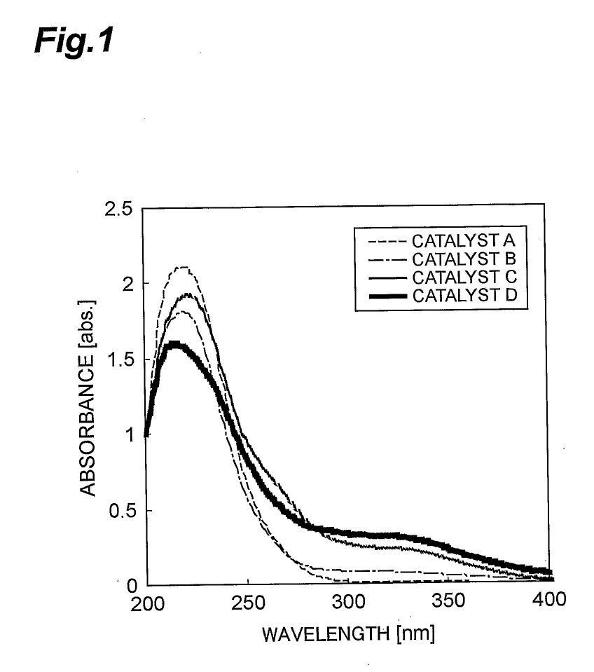

In the meantime, UV-visible absorption spectra of Titanosilicate Catalysts A to D obtained in Example 1 and Comparative Examples 1 and 2 were measured. In measuring the UV-visible absorption spectra, the samples were pulverized well in an agate mortar and filled in sample cells (inner diameter, 10 mm φ; depth, 3 mm) in such a way that the surfaces became flat. Measurements were made under the following conditions. Reflectance was transformed by K-M conversion into absorbance (abs.), which was corrected so that the absorbance (abs.) at 200 mn became 1. The results are shown in FIG. 1.

<Conditions for Measurement of UV-Visible Absorption Spectra>

### Measuring Equipment

(main body): UV-visible spectrophotometer (V-7100, produced by JASCO Corp.)

(attachment): diffuse reflectance apparatus (Praying Mantis, produced by Harrick)
Pressure: atmospheric pressure
Measured value: reflectance
Data collection time: 0.1 second
Band width: 2 nm
Measured wavelength:200 to 400 nm
Slit height: half open
Data collection interval: 1 nm
Baseline correction (reference): standard white plate (Spectralon)

FIG. 1 is a graph showing the results of measurement of the UV-visible absorption spectra of titanosilicates obtained in Example 1 and Comparative Examples 1 and 2. It is clear from FIG. 1 that the titanosilicates obtained in Example 1 show, in comparison to the titanosilicates obtained in Comparative Examples 1 and 2, more absorption at around 210 nm to 230 nm, which represents 4-coordinated Ti species, and less absorption at 320 nm to 330 nm, which represents Ti species outside the framework.

1. A layered titanosilicate obtained by contacting a layered borosilicate with a titanium source and an inorganic acid.

2. The layered titanosilicate according to claim 1, having an X-ray diffraction pattern with the following values and also having a composition represented by the general formula: xTiO₂.(1-x)SiO₂ (in the formula, x represents a number from 0.0001 to 0.1);
X-ray diffraction pattern:
(Lattice spacing d/Å)
12.3±0.3
11.0±0.3
9.0±0.3
6.1±0.3
3.9±0.2
3.4±0.1

3. The layered titanosilicate according to claim 1, wherein the inorganic acid is nitric acid.

4. A titanosilicate having a zeolite structure, obtained by heat-treating the layered titanosilicate according to claim 1.

5. The titanosilicate having a zeolite structure according to claim 4, having an X-ray diffraction pattern with the following values and also having a composition represented by the general formula: xTiO₂.(1-x)SiO₂ (in the formula, x represents a number from 0.0001 to 0.1);
X-ray diffraction pattern:
(Lattice spacing d/Å)
12.3±0.3
11.0±0.3
9.0+0.3
6.1±0.3
3.9±0.2
3.4±0.1

6. A silylated layered titanosilicate, obtained by silylating the layered titanosilicate according to claim 1.

7. A silylated titanosilicate having a zeolite structure, obtained by silylating the layered titanosilicate having a zeolite structure according to claim 4.

8. A method for producing a layered titanosilicate, comprising of contacting a layered borosilicate with an inorganic acid and a titanium source.

9. The method for producing a layered titanosilicate according to claim 8, wherein the inorganic acid is nitric acid.

10. A method for producing an epoxy compound, including a step of reacting an olefin, oxygen and hydrogen in the presence of at least one titanosilicate and a noble metal catalyst, the titanosilicate being selected from a group consisting of the layered titanosilicate according to claim 1.

11. The method for producing an epoxy compound according to claim 10, wherein a nitrile compound is used as a solvent.
